(19) United States
(12) Patent Application Publication
(10) Pub. No.: US 2006/0217774 A1
(43) Pub. Date: Sep. 28, 2006

(54) Cardiac contractile augmentation device and method therefor

(76) Inventors: Morton M. Mower, Baltimore, MD (US); Jon L. Roberts, Great Falls, VA (US); Elliott D. Light, Rockville, MD (US)

Correspondence Address: ROBERTS ABOKHAIR & MARDULA, SUITE 1000, 11800 SUNRISE VALLEY DRIVE, RESTON, VA, 20191, US

(21) Appl. No.: 11/442,519

(22) Filed: May 26, 2006

Related U.S. Application Data
(63) Continuation-in-part of application No. 11/141,403, filed on May 31, 2005.
Application No. 11/141,403 is a continuation-in-part of application No. 10/053,750, filed on Jan. 21, 2002.
Application No. 10/053,750 is a continuation of application No. 09/690,947, filed on Oct. 18, 2000, now Pat. No. 6,341,235.
Application No. 09/690,947 is a continuation-in-part of application No. 09/008,636, filed on Jan. 16, 1998, now Pat. No. 6,136,019.
Application No. 09/008,636 is a continuation-in-part of application No. 08/699,552, filed on Aug. 19, 1996, now Pat. No. 5,871,506.
(60) Provisional application No. 60/691,444, filed on Jun. 17, 2005.

(51) Int. Cl. A61N 1/362

(52) U.S. Cl. 607/9; 600/16

(57) ABSTRACT

A cardiac contractile augmentation device (CCAD) comprising an EAP segment adapted to be attached to a portion of the heart that would benefit from contractile augmentation. The EAP segment is energized by a pulse generator. In response to an electrical pulse the EAP segment deforms resulting in a contraction of the portion of the heart to which the EAP fabric is attached. In response to another pulse from the pulse generator, the EAP segments returns to its pre-deformed state. A CCAD may be constructed of EAP segments that are independently energized under control of a processor that is connected to the individual segments. The processor causes the pulse generator to sequentially pulse the individual segments in the direction of a normal contraction of the cardiac tissue. In this way, the CCAP provides contractile augmentation to a chamber of the heart in a pattern that is equivalent to a concentric contraction of normal heart muscle. Sensors may be used to provide the processor data indicative of the state of the heart and the need for contractile augmentation. The CCAD may be configured as a cardiac patch, a cardiac wrap, or a cardiac envelope. The EAP segments may be energized during systole to augment the contractile strength of the heart. The EAP segments may also be energized during diastole to provide a passive restraint precisely calibrated to the degree of restraint desired. Moreover, the active cardiac envelope may be energized during both systole and diastole. Networks of EAP sensors and contractile device may be use to provide the sensing and contractile functionality. Alternatively, a dual function EAP may perform both sensing and contractile functions.

### CROSS REFERENCE TO RELATED APPLICATIONS

This application claims the benefit under 35 U.S.C. § 119(e) from provisional application No. 60/691,444 filed Jun. 17, 2005. The 60/691,444 provisional application is incorporated by reference herein, in its entirety, for all purposes. This application is a continuation in part of U.S. patent application Ser. No. 11/141,403, filed May 31, 2005, pending, which is a continuation in part of U.S. patent application Ser. No. 10/053,750 filed Jan. 21, 2002, pending, which is a continuation of U.S. patent application Ser. No. 09/690,947, filed Oct. 18, 2000, now U.S. Pat. No. 6,341,235, which is a continuation-in-part of U.S. patent application Ser. No. 09/008,636 filed Jan. 16, 1998, now U.S. Pat. No. 6,136,019, which is a continuation-in-part of U.S. patent application Ser. No. 08/699,552, filed Aug. 19, 1996, now U.S. Pat. No. 5,871,506. The Ser. Nos. 11/141,403, 10/053,750, 09/690,947, 09/008,636, and 08/699,552 applications, and U.S. Pat. Nos. 6,341,235, 6,136,019, and 5,871,506 are all incorporated by reference herein, in their entirety, for all purposes.

### BACKGROUND

The present invention pertains to a device and method for treating heart disease. More particularly, the present invention is directed to a method and device for treating diseases and injuries to the heart that are associated with adverse cardiac remodeling.

### Remodeling

The heart is divided into the right side and the left side. The right side, comprising the right atrium and ventricle, collects and pumps de-oxygenated blood to the lungs to pick up oxygen. The left side, comprising the left atrium and ventricle, collects and pumps oxygenated blood to the body. Oxygen-poor blood returning from the body enters the right atrium through the vena cava. The right atrium contracts, pushing blood through the tricuspid valve and into the right ventricle. The right ventricle contracts to pump blood through the pulmonic valve and into the pulmonary artery, which connects to the lungs. The blood picks up oxygen in the lungs and then travels back to the heart through the pulmonary veins. The pulmonary veins empty into the left atrium, which contracts to push oxygenated blood into the left ventricle. The left ventricle contracts, pushing the blood through the aortic valve and into the aorta, which connects to the rest of the body. Coronary arteries extending from the aorta provide the heart blood.

The heart's own pacemaker is located in the atrium and is responsible for initiation of the heartbeat. The heartbeat begins with activation of atrial tissue in the pacemaker region (i.e., the sinoatrial or “SA” node), followed by cell-to-cell spread of excitation throughout the atrium. The only normal link of excitable tissue connecting the atria to the ventricles is the atrioventricular (AV) node located at the boundary between the atria and the ventricles. Propagation takes place at a slow velocity, but at the ventricular end the bundle of His (i.e., the electrical conduction pathway located in the ventricular septum) and the bundle branches carry the excitation to many sites in the right and left ventricle at a relatively high velocity of 1-2 m/s. The slow conduction in the AV junction results in a delay of around 0.1 seconds between atrial and ventricular excitation. This timing facilitates terminal filling of the ventricles from atrial contraction prior to ventricular contraction. After the slowing of the AV node, the bundle of His separates into two bundle branches (left and right) propagating along each side of the septum. The bundles ramify into Purkinje fibers that diverge to the inner sides of the ventricular walls. This insures the propagation of excitatory pulses within the ventricular conduction system proceeds at a relative high speed when compared to the propagation through the AV node.

Remodeling of the heart is a harmful physical change in the heart that occurs with heart failure, heart attack, and heart disease. Remodeling is characterized by enlargement of the heart and thinning of the heart walls. For example, after a heart attack, while the normal heart muscle responds normally to excitatory pulses. tissue that is damaged by the heart attack does not respond or responds in a slower than normal rate to excitatory pulses. The healthy tissue however, continuing to function normally, places increased stress on the damaged and marginalized tissue, thereby “stretching” it. The stretching increases the volume of blood held by the heart resulting in a short term increased blood output via a Frank-Sterling mechanism. In this way, the heart muscle behaves something like a rubber band—the more it is stretched, the more “snap” the heart generates. However, if cardiac muscle is overstretched, or if the heart is stretched repetitively over a long period of time, it eventually loses its “snap” and becomes flaccid (a form of remodeling). Remodeling progresses in stages. Following a heart attack or as a consequence of heart disease, the heart becomes rounder and larger. Heart muscle cells die and the heart as a pump gets weaker. If the remodeling is allowed to progress, the heart's main pumping chamber—the left ventricle—enlarges and changes shape, getting rounder. The heart also undergoes changes at the cell level.

### Heart Failure

The syndrome of “heart failure” is a common course for the progression of many forms of heart disease. Heart failure may be considered to be the condition in which an abnormality of cardiac function is responsible for the inability of the heart to pump blood at a rate commensurate with the requirements of the metabolizing tissues, or can do so only at an abnormally elevated filling pressure. Typically, the elevated filling pressures result in dilatation of the left ventricular chamber. Etiologies that can lead to this form of failure include idiopathic cardiomyopathy, viral cardiomyopathy, and ischemic cardiomyopathy.

Heart failure is a chronic condition that affects over five million Americans, and is the most common reason for hospitalization among elderly persons. Contrary to its name, heart failure is not a heart attack, although heart attacks can lead to heart failure. Neither does the heart suddenly stop beating. Heart failure means that the heart is failing to pump enough blood to meet the body's needs. It often occurs in patients whose hearts have been weakened or damaged by a heart attack or other conditions. As the heart continues to fail, patients may experience breathlessness, fluid build-up in the limbs and severe fatigue. Delays in response of the septum to excitatory pulse may cause contractions that are not simultaneous and therefore the ventricular contraction pattern is non-concentric. In this mode, the heart is beating inefficiently.

When the heart is working properly, both of its lower chambers (ventricles) pump at the same time and in sync with the pumping of the two upper chambers (atria). Up to 40 percent of heart failure patients, however, have disturbances in the conduction of electrical impulses to the ventricles (e.g., bundle branch block or intraventricular conduction delay). As a result, the left and right ventricles are activated at different times. When this happens, the walls of the left ventricle (the chamber responsible for pumping blood throughout the body) do not contract simultaneously, reducing the heart's efficiency as a pump. The heart typically responds by beating faster and dilating. This results in a vicious cycle of further dilation, constriction of the vessels in the body, salt and water retention, and further worsening of heart failure. These conduction delays do not respond to antiarrhythmics or other drugs.

Patients who have heart failure may be candidates to receive a pacemaker. A biventricular pacemaker is a type of implantable pacemaker designed to treat heart failure. A biventricular pacemaker can help synchronize the lower chambers by sending electrical signals simultaneously to the left ventricle and to the right ventricle. By stimulating both ventricles (biventricular pacing), the pacemaker makes the walls of the right and left ventricles pump together again, and cause the septum to be stabilized and be part of the contraction pattern for each ventricle. The heart is thus resynchronized, pumping more efficiently while causing less wear and tear on the heart muscle itself. This is why biventricular pacing is also referred to as cardiac resynchronization therapy (CRT).

For patients who suffer from heart failure, remodeling of the heart may occur. Remodeling associated with heart failure is characterized by enlargement of the heart's left ventricle. In addition, the left ventricle walls become thinner. There is an increased use of oxygen, greater degree of mitral valve regurgitation, and decreased ejection fraction. Remodeling sets off a “domino effect” of further damage to heart cells and more severe heart disease. Biventricular pacing of the present invention can potentially reverse the process. This beneficial effect on the heart is called “reverse remodeling.” Typical biventricular pacemakers use cathodal pulses of 2.5 volts in the atrium and 5 volts in the ventricle.

### Heart Attack

A heart attack is an event that results in permanent heart damage or death. It is also known as a myocardial infarction, because part of the heart muscle (myocardium) may literally die (infarct). A heart attack occurs when one of the coronary arteries becomes severely or totally blocked, usually by a blood clot. When the heart muscle does not receive the oxygenated blood that it needs, it will begin to die. The severity of a heart attack usually depends on how much of the heart muscle is injured or dies during the heart attack.

Although a heart attack is usually the result of a number of chronic heart conditions (e.g., coronary artery disease), the trigger for a heart attack is often a blood clot that has blocked the flow of blood through a coronary artery. If the artery has already been narrowed by fatty plaque (a disease called atherosclerosis), the blood clot may be large enough to block the blood flow severely or completely. The victim may experience an episode of cardiac ischemia, which is a condition in which the heart is not getting enough oxygenated blood. This is often accompanied by angina (a type of chest pain, pressure or discomfort), although silent ischemia shows no signs at all. Severe or lengthy episodes of cardiac ischemia can trigger a heart attack. Depending upon the severity of both the attack and of the subsequent scarring, a heart attack can lead to the following:
Heart failure, a chronic condition in which at least one chamber of the heart is not pumping well enough to meet the body's demands.
Electrical instability of the heart, which can cause a potentially dangerous abnormal heart rhythm (arrhythmia).
Cardiac arrest, in which the heart stops beating altogether, resulting in sudden cardiac death in the absence of immediate medical attention.
Cardiogenic shock, a condition in which damaged heart muscle cannot pump normally and enters a shock-like state that is often fatal.
Death.

Whether or not the heart muscle will continue to function after a heart attack depends on how much of it was damaged or how much of it died before the patient could get medical treatment. The location of the damage in the heart muscle is also important. Because different coronary arteries supply different areas of the heart, the severity of the damage will depend upon the degree to which the artery was blocked and the amount and area of the heart muscle that depended on that blocked artery.

As previously noted, tissue that is damaged by the heart attack does not respond or responds a slower than normal rate to excitatory pulses. The healthy tissue operates normally, but as a consequence places increased stress on this marginalized tissue, thereby “stretching” it. It is desirable to treat a heart attack so as to minimize the likelihood of continued detrimental remodeling. This can be accomplished by reducing the contraction strength of healthy cardiac tissues, by increasing the contraction strength of marginalized cardiac tissue, or by implementing a combination of both therapies.

### Congestive Heart Failure

Congestive heart failure (CHF) (also referred to as congestive heart disease) is a progressive and debilitating illness. The disease is characterized by a progressive enlargement of the heart and a concomitant weakening of the heart's weak pumping action. This weakened pumping action results in a buildup of fluid called congestion in the lungs and other body tissues. The heart tries to make up for this weakening by enlarging and by pumping faster to move more blood through the body.

As the heart enlarges, (sometimes referred to as “adverse remodeling”), the heart performs an increasing amount of work in order to pump blood each heart beat. In time, the heart becomes so enlarged it cannot adequately supply blood. An afflicted patient is fatigued, unable to perform even simple exerting tasks and experiences pain and discomfort. Further, as the heart enlarges, the internal heart valves cannot adequately close. This impairs the function of the valves and further reduces the heart's ability to supply blood.

Causes of congestive heart disease are not fully known. In certain instances, congestive heart disease may result from viral infections. In such cases, the heart may enlarge to such an extent that the adverse consequences of heart enlargement continue after the viral infection has passed and the disease continues its progressively debilitating course.

Patients suffering from congestive heart disease are commonly grouped into four classes (i.e., Classes I, II, III and IV). In the early stages (e.g., Classes I and II), drug therapy is the commonly proscribed treatment. Drug therapy treats the symptoms of the disease and may slow the progression of the disease. Importantly, there is no cure for congestive heart disease. Even with drug therapy, the disease will progress. Further, the drugs may have adverse side effects.

Presently, the only permanent treatment for congestive heart disease is heart transplant. To qualify, a patient must be in the later stage of the disease (e.g., Classes III and IV with Class IV patients given priority for transplant). Such patients are extremely sick individuals. Class III patients have marked physical activity limitations and Class IV patients are symptomatic even at rest.

Due to the absence of effective intermediate treatment between drug therapy and heart transplant, Class III and IV patients will have suffered terribly before qualifying for heart transplant. Further, after such suffering, the available treatment is unsatisfactory. Heart transplant procedures are very risky, extremely invasive and expensive and only extend a patient's life for a relatively short period of time. For example, prior to transplant, a Class IV patient may have a life expectancy of 6 months to one-year. Heart transplant may improve the expectancy to about five years.

Unfortunately, not enough hearts are available for transplant to meet the needs of congestive heart disease patients. In the United States, in excess of 35,000 transplant candidates compete for only about 2,000 transplants per year. A transplant waiting list is about 8-12 months long on average and frequently a patient may have to wait about 1-2 years for a donor heart. While the availability of donor hearts has historically increased, the rate of increase is slowing dramatically. Even if the risks and expense of heart transplant could be tolerated, this treatment option is becoming increasingly unavailable. Further, many patient's do not qualify for heart transplant for failure to meet any one of a number of qualifying criteria

Congestive heart failure has an enormous societal impact. In the United States alone, about five million people suffer from the disease. According to the American Heart Association, people 40 and older have a 1 in 5 chance of developing CHF in their lifetime. Nearly 5 million people in the United States—mostly older adults—already have CHF, and the number of people with CHF keeps rising. About 550,000 people develop CHF each year. This is because people are living longer and surviving heart attacks and other medical conditions that put them at risk for CHF. People who have other types of heart and vessel disease are also at risk for CHF. The economic costs of the disease have been estimated at $38 billion annually. Not surprising, substantial effort has been made to find treatments for congestive heart disease.

Typically, many treatments for CHF are directed to easing the workload of the heart. Treatment may include lifestyle changes, medicines, transcatheter interventions, and surgery.

Studies show that medicines may help improve the heart function. Diuretics, help rid your body of extra fluid. Inotropics, such as digitalis strengthen the heart's ability to pump. Vasodilators, such as nitroglycerin, open up narrowed vessels. Calcium channel blockers, ACE inhibitors, and Angiotensin II receptor blockers keep vessels open and lower blood pressure. Beta blockers, which have been shown to help increase your ability to exercise and improve your symptoms over time.

Surgical treatments are also used to stop or slow the progression of CHF. Angioplasty is a procedure that is used to open arteries narrowed by fatty plaque buildup. It is performed in a cardiac catheterization laboratory. Doctors use a long, thin tube called a catheter that has a small balloon on its tip. They inflate the balloon at the blockage site in the artery to flatten the fatty plaque against the artery wall.

Stenting is used along with balloon angioplasty. It involves placing a mesh-like metal device into an artery at a site narrowed by plaque. The stent is mounted on a balloon-tipped catheter, threaded through an artery, and positioned at the blockage. The balloon is then inflated, opening the stent. Then, the catheter and deflated balloon are removed, leaving the stent in place. The opened stent keeps the vessel open and stops the artery from collapsing.

Cardiomyoplasty is one potential treatment for moderate stage congestive heart disease. In this procedure, the latissimus dorsi muscle (taken from the patient's shoulder) is wrapped around the heart and chronically paced to assist contraction of the heart during systole.

One study speculates that an artificial elastic “sock” could be used in place of the latissimus dorsi in adynamic cardiomyoplasty. See, Kass et al., “Reverse Remodeling from Cardiomyoplasty in Human Heart Failure,” Circulation 91:9, May 1, 1995 (herein, “Kass”).

Another study demonstrates that Bard Marlex sheets can be used to wrap the heart as a substitute to the latissimus dorsi in adynamic cardiomyoplasty. See, Oh et al., “The Effects of Prosthetic Cardiac Binding and Adynamic Cardiomyoplasty in a Model of Dilated Cardiomyoplasty,” Journal of Thoracic Cardiovascular Surgery, 116:1, July 1998. (Herein, “Oh.”)

German Utility Model Patent Application DE 295 17 393 U1 describes a pericardium prosthesis made from a biocompatible, non-expansible material, or at least hardly expansible material that surrounds the heart to prevent over-expansion of the heart wall. (Herein, the “DE '393 application.”)

PCT application WO 98/58598 describes an elastic pouch for at least partially enveloping a heart. (Herein, the PCT '598 application.”)

Commonly assigned U.S. Pat. No. 5,702,343 to Alferness dated Dec. 30, 1997 teaches a envelope to constrain cardiac expansion during diastole. (Herein, U.S. Pat. No. '343.) Other teachings include those of commonly assigned U.S. Pat. No. 6,123,662 (herein, U.S. Pat. No. '662) and those of U.S. Pat. Application Publication No. US 2002/0019580 (herein, “USApp 19580.”)

U.S. Pat. Application Publication No. U.S. 2002/0103511 filed by Alferness, et al. for a “Defibrillating Cardiac Constraint” (herein, “USApp 103511”) describes an envelope of flexible material for placing over the heart. The envelope is dimensioned for an apex of the heart to be inserted into the volume through the open upper end and for the envelope to be slipped over the heart. The envelope is adapted to be secured to the heart with the envelope having portions disposed on opposite sides of the heart. The envelope is adjustable to snugly conform to an external geometry of the heart and to constrain circumferential expansion of the heart during diastole and permit substantially unimpeded contraction of the heart during systole. Grids of electrodes are carried on the envelope. The grids are disposed to be in overlying relation to individual ones of the opposite sides of the heart when the envelope is secured to the heart. The grids are independently connectable to a source of a defibrillating waveform.

The Kass and Oh publications are hereby incorporated herein by reference in their entirety for all purposes. The DE '393 application, the PCT '598 application, U.S. Pat. No. '343, U.S. Pat. No. '662, USApp 19580, and USApp 103511 are also incorporated by reference herein in their entirety for all purposes.

Passively constraining the heart, while limiting cardiac enlargement, does little to relieve the underlying weakness of the heart's pumping action. Pacing the heart electrically may increase the stroke volume but relies on cardiac muscles that may have been weakened by the disease itself or by other illnesses.

It has been suggested that electroactive polymers (EAPs) be configured for use as artificial muscles. EAPs can be deformed repetitively by applying external voltage across the EAP, and they can quickly recover their original configuration upon reversing the polarity of the applied voltage. An EAP has a high load bearing capacity to mass ratio, short response time, and nearly linear deformation response with respect to applied voltage. Artificial muscle polymers can be formed from a conductive polymer doped with surfactant molecule or from an ionic polymer metal composite (IPMC). Doped electroactive polymers (EAPs) are conductive polymers (e.g., polypyrrole or polyanaline) doped with a surfactant (e.g., sodium dodecyl benzene sulfonate). IPMCs typically consist of perfluorsulfonate polymers that contain small proportions of sulfonic or carboxyic ionic functional groups. Nafion®, a polymer made by DuPont, is one example of a poly(tetrafluoroethylene) based ionomer.

For its application as an artificial muscle, Nafion® can be produced in a sheet geometry with positive counter ion (e.g., Na+ or Li+) contained in the matrix. The outer surface region (less than a micrometer) of the polymer sheet is then impregnated with a conductive metal such as platinum or gold. The resulting EAP absorbs water until its physical ability to expand is balanced by the affinity of water for the polymer-fixed ions and free counter ions. When an electrical field is applied across the EAP, the EAP deforms as a result of stresses generated by the movement of water and mobile positive ions in the polymer composite.

What is needed is a system and method for treating the causes of CHF that provides improvement in the pumping strength of the heart. Such a system and method would further aid in correcting heart enlargement, whether as a distinct objective or in conjunction with improving the pumping strength of the heart. Embodiments of the present invention fulfill these needs by utilizing an EAP fabric formed to provide contractile augmentation to selected areas of the heart.

### SUMMARY

An embodiment of the present invention a cardiac contractile augmentation device (CCAD) comprises an EAP segment adapted to be attached to a portion of the heart that would benefit from contractile augmentation. In this embodiment, the EAP segment is energized by a pulse generator. In response to an electrical pulse the EAP segment deforms resulting in a contraction of the portion of the heart to which the EAP fabric is attached.

In another embodiment of the present invention, a CCAD is constructed of EAP segments that independently energized. A pulse generator under control of a processor is connected to the individual segments. The processor causes the pulse generator to sequentially pulse the individual segments in the direction of a normal contraction of the cardiac tissue. In this way, the CCAD provides contractile augmentation to a chamber of the heart in a pattern that is equivalent to a concentric contraction of normal heart muscle.

In still another embodiment of the present invention, two EAP segments are arranged back-to-back. The EAP segments are then energized with different polarities such that one relaxes the other contracts along the same axis. In even another embodiment of the present invention, two EAP segments are overlapped at right angles to each other provide a global contraction pattern.

In another embodiment of the present invention sensors are used to provide a processor data indicative of the state of the heart and the need for contractile augmentation. Sensors may be attached to the heart or may be integrated into a CCAD. Sensors may be used to detect the activation of the heart or to detect a measure of heart activity. By way of illustration and not as a limitation, sensing may be directed to metabolic demand, the QT interval of the ECG cycle, the right ventricular systolic pressure blood oxygen saturation, the respiration rate partial pressure of carbon dioxide in the blood, blood temperature and pre-ejection period.

In still another embodiment, the CCAD comprises a dual use polymeric blend for both sensing and contractile purposes. Such polymeric blends are described in U.S. Pat. No. 6,689,288 which is incorporated herein by reference in its entirety for all purposes. In this embodiment, the CCAD senses when and where contractions may be required. A processor connected to the CCAD then directs current to the appropriate region of the CCAD to cause contractions to occur thereby providing mechanical assistance to the heart.

In still another embodiment of the present invention, a CCAD comprises EAP fabric that surrounds the heart (herein referred to as an “active cardiac envelope”). According to this embodiment, the active cardiac envelope comprises EAP segments integrated with a highly compliant and elastic device constructed from a biocompatible material that is applied to an external surface of a heart. The elastic material resists dilatation of the heart, provides acute wall support, and enhances reduction in the size of the heart using stored potential energy. The EAP segments may be energized during systole to augment the contractile strength of the heart. The EAP segments may also be energized during diastole to provide a “passive restraint” precisely calibrated to the degree of restraint desired. Moreover, the active cardiac envelope may be energized during both systole and diastole. Once again, dual use (sensing-contraction) polymeric blends may be used to construct the active cardiac envelope.

Thus, the size of the active cardiac envelope may be adjusted automatically as the heart size changes by using different “bias currents.” In an embodiment of the present invention, the bias voltage is applied as a “chopped” waveform with an asymmetric “duty cycle” to avoid any physical changes that the current might induce in the EAP material (and also to reduce the overall current drain) without interfering with systolic contraction.

It is therefore an aspect of the present invention to augment the contraction of a selected portion of the heart.

It is another aspect of the present invention to emulate the normal concentric contraction of a selected portion of the heart using a CCAD comprising one or more EAP segments.

It is still another aspect of the present invention to use sensors to acquire data indicative of the state of the heart and the need for contractile augmentation.

It is yet another aspect of the present invention to incorporate sensors into a CCAD.

It is an aspect of the present invention to drive a pulse generator based on sensor data and to apply electrical stimulation to EAP segments of the CCAD to provide contractual augmentation active to the sensor data.

It is another aspect of the present invention to apply an active cardiac envelope to the heart to limit the outward expansion of the heart.

It is yet another aspect of the present invention to control the active cardiac envelope in response to sensor data.

It is still another aspect of the present invention to augment pacemaker stimulation of the heart using a CCAD comprising one or more EAP segments.

It is another aspect of the present invention to apply biphasic electrical pacing and mechanical pacing using a CCAD to the heart wherein the biphasic pacemaker and the CCAD are responsive to sensor data.

In is yet another aspect of the present invention to apply biphasic, biventricular or left ventricle stimulation to undamaged areas of the heart and to apply mechanical pacing using a CCAD to enhance the muscular contraction of the healthy tissue thereby allowing the heart to achieve normal or near-normal functioning thereby preventing or reducing the adverse forms of remodeling of the heart following a myocardial infarction.

It is another aspect of the present invention to apply to the biphasic, biventricular stimulation continuously to the right and left ventricles through electrodes that contact undamaged portions of the heart.

In an embodiment of the present invention, a cardiac contractile augmentation device (CCAD) comprise an electroactive polymer (EAP) linear segment attached along its length to a selected area of a heart. The EAP segment may be energized during systole to augment the contractile strength of the heart. By way of illustration and not as a limitation, the selected area of the heart is may be ventricle or an atrium.

In another embodiment of the present invention, the CCAD further comprises a pulse generator. The pulse generator generates a contraction electrical signal that coincides with an excitatory pulse directed to the heart at systole and generates a relaxation signal that coincides with a refractory period of the heart at diastole. The EAP segment receives the contraction signal from the pulse generator. In response to the contraction signal from the pulse generator, the EAP segment contracts along its length thereby augmenting a contraction of the selected area of the heart to which the EAP segment is attached. In response to the relaxation signal from the pulse generator, The EAP segment returns to a relaxed state.

In yet another embodiment of the present invention, the CCAD further comprises a sensor and a processor. The sensor acquires sensor data indicative of the state of the heart. The processor determines from the sensor data the need for contractile augmentation of the selected area. The pulse generator is responsive to commands from the processor and provides contraction and relaxation electrical signals.

In still another embodiment of the present invention, the sensor data comprises at least one measure of the state of the heart selected from the group consisting of a measure of metabolic demand, an occurrence of the QT interval of the ECG cycle, a measure of a right ventricular systolic pressure blood oxygen saturation, a measure of a respiration rate partial pressure of carbon dioxide in the blood, a measure of blood temperature, and an occurrence of a pre-ejection period.

In an embodiment of the present invention, the contraction electrical signal comprises a chopped waveform with an asymmetric duty cycle.

In yet another embodiment of the present invention, the excitatory pulse may be generated by the heart or by a heart stimulation device.

In still another embodiment of the present invention, the heart stimulation device comprises a left ventricular electrode group, wherein the left ventricular electrode groups comprise LV electrodes attached to the left ventricle at increasing distances from the AV node and a right ventricular electrode group, wherein the right ventricular electrode group comprises RV electrodes attached to the left ventricle at increasing distances from the AV node. The heart stimulation device attaches to the LV and RV electrodes. The heart stimulation device generates a timing signal coincident with a refractory period of the heart. In response to the timing signal, the heart stimulation device sends pulses to the LV and RV electrodes sequenced such that an initial pulse arrives at an LV electrode and at an RV electrode nearest the AV junction and subsequent pulses arrive at an LR and at an RV electrode progressively further from the AV junction.

In an embodiment of the present invention, a cardiac contractile augmentation device (CCAD) comprises two or more electroactive polymer (EAP) linear segments each attached along its length to a selected area of a heart. Selected EAP segments may be energized during systole to augment the contractile strength of the heart.

In an embodiment of the present invention, an active cardiac envelope (ACE) comprises a biomedical material that can be applied to the epicardial surface of the heart and an augmentation EAP segment integrated with the biomedical material. The biomedical material expands to a predetermined size, the predetermined size selected to constrain cardiac expansion beyond a predetermined limit. The augmentation EAP segment may be energized during systole to augment the contractile strength of the heart.

In another embodiment of the present invention, the ACE surrounds the epicardial surface of the heart and circumferentially constrains cardiac expansion. In this embodiment, the envelope has a base end, the base end having an opening for applying the envelope to the epicardial surface of the heart by passing the envelope over the epicardial surface of the heart such that when applied to the epicardial surface, the base end of the envelope is oriented toward the base of the heart. The envelope also has an apex end such that when the envelope is applied to the epicardial surface, the apex end is oriented towards the apex of the heart. The base end of the envelope comprises a securing arrangement for securing the envelope to the epicardial surface of the heart.

In yet another embodiment of the present invention, the ACE further comprises a sensor, a processor, and a pulse generator. The sensor acquires sensor data indicative of the state of the heart. The processor determines from the sensor data the need for contractile augmentation of the selected area. The pulse generator responds to a command from the processor to provide a contraction signal to the augmentation EAP segment.

In an embodiment of the present invention, a cardiac contractile augmentation device (CCAD) comprises an electroactive polymer (EAP) linear segment attached along its length to a selected area of a heart and an EAP pulse generator. The EAP segment may be energized during systole to augment the contractile strength of the heart. The EAP pulse generator generates a contraction electrical signal that coincides with the application of a biphasic pacing pulse from a heart stimulation device connected to the heart, and generates a relaxation signal that coincides with a refractory period of the heart at diastole. The EAP linear segment receives the contraction signal from the pulse generator and in response to the contraction signal from the pulse generator contract along its length thereby augmenting a contraction of the selected area of the heart to which the EAP segment is attached. In response to the relaxation signal from the EAP pulse generator, the EAP segment returns to a relaxed state.

In yet another embodiment of the present invention, the CCAD further comprises a sensor and a processor. The sensor acquires sensor data indicative of the state of the heart. The processor determines from the sensor data the need for contractile augmentation of the selected area. The EAP pulse generator is responsive to commands from the processor to provide contraction and relaxation electrical signals.

In an embodiment of the present invention, an active cardiac fabric (ACF) system comprises a pulse generator, a processor, and an ACF. The pulse generator is responsive to the processor. The ACF comprises a network of EAP sensors and a network of EAP contractile devices that can be applied to a selected segment of the heart. An EAP sensor senses electrical activity of the heart and generates an electrical activity signal in response to the sensed activity. An EAP contractile device contracts in response to a contraction signal sent by the pulse generator. The processor receives the electrical activity signal from the EAP sensor and determines whether the heart requires contractile augmentation at location proximate to the EAP contractile device. The processor sends a contraction instruction to the pulse generator to generate a contraction signal if the heart requires contractile augmentation at the location proximate to the EAP contractile device. The pulse generator sends the contraction signal to the EAP contractile device.

In another embodiment of the present invention, an active cardiac fabric (ACF) system comprises a pulse generator, a processor, and an active cardiac fabric comprising a network of dual function EAP device that can be applied to a segment of the heart. The pulse generator is responsive to the processor. A dual function EAP device senses electrical activity of the heart and generates an electrical activity signal in response to the sensed activity. The dual function EAP device contracts in response to a contraction signal sent by the pulse generator. The processor receives the electrical activity signal and determines whether the heart requires contractile augmentation at location proximate to the dual function EAP. The processor sends a contraction instruction to the pulse generator to generate a contraction signal if the heart requires contractile augmentation at the location proximate to the dual function EAP. The pulse generator sends the contraction signal to the dual function EAP.

### DESCRIPTION OF THE DRAWINGS

FIG. 1 illustrates an EAP segment according to the prior art.

FIG. 2 illustrates a cardiac contractile augmentation device comprising an EAP segment that is applied to a selected area of the heart according to an embodiment of the present invention.

FIG. 3 illustrates a cardiac contractile augmentation device comprising a plurality of EAP segments that is applied to a selected area of the heart according to an embodiment of the present invention.

FIG. 4 illustrates a cardiac contractile augmentation device configured as a cardiac wrap and comprising at least one EAP segment according to an embodiment of the present invention.

FIG. 5 a cardiac contractile augmentation device configured as a cardiac envelope and comprising at least one EAP segment according to an embodiment of the present invention.

FIG. 6 illustrates two EAP segments arranged back-to-back according to an embodiment of the present invention.

FIG. 7 illustrates two EAP segments that are overlapped according to an embodiment of the present invention.

FIG. 8 illustrates a network of dual (sensing-contraction) use EAPs to form the fabric of a CCAD according to an embodiment of the present invention.

FIG. 9 illustrates a sensing network and a contracting network of EAPs which form the fabric of the CCAD according to an embodiment of the present invention.

### DETAILED DESCRIPTION

The following terms are used in the description that follows. The definitions are provided for clarity of understanding:
Concentric When a muscle is activated and required to lift a load which is less than the maximum tetanic tension it can generate, the muscle begins to shorten. Contractions that permit the muscle to shorten are referred to as concentric contractions. An example of a concentric contraction in the raising of a weight during a bicep curl.
EAP electroactive polymers
ACE inhibitor Angiotensin Converting Enzyme inhibitors—A type of heart failure medication that works by preventing the body from creating angiotensin, a substance in the blood that causes vessels to tighten and raises blood pressure. In large-scale studies, ACE inhibitors have been proven to slow the progression of heart failure.
Angioplasty A procedure that reopens blocked blood vessels to the heart. A physician inserts a hollow needle (catheter) into the diseased artery and pushes a small deflated balloon into the blocked section. Then the physician inflates the balloon to widen the artery.
Angiotensin II receptor blocker A medication that blocks the action of a special chemical called angiotensin, which normally raises the heart rate and blood pressure.
Arrhythmia An abnormal rhythm or rate of the heartbeat caused by disturbances in the movement of electrical impulses through the heart.
Atrial fibrillation Rapid, uneven contractions in the upper heart chambers (atria), which cause the lower chambers (ventricles) to beat irregularly.
Atrium One of the two upper chambers of the heart.
Beta blockers Medications that reduce the heart's tendency to beat faster by blocking specific receptors (“beta receptors”) on the cells that make up the heart.
Blood thinners Medications, such as warfarin and heparin, used to prevent blood clotting. Some people with heart failure are prescribed blood thinners to reduce the risk of stroke.
Calcium channel blocker A drug that prevents calcium from entering the heart's muscle cells. This causes the muscles to relax, lowering the heart rate.
Cardiac rehabilitation A supervised program of increasing exercise, mental support and training to allow a person with a heart condition to resume normal activities.
Cardiomyopathy Any weakening or deformity of the heart muscle that causes decreased pumping force. This leads to less-efficient circulation of blood through the lungs and the rest of the body.
Cardiomyoplasty An investigational surgical treatment for heart failure that involves taking muscles from the person's back or abdomen and wrapping them around the heart. Its goal is to increase the heart's pumping power.
Congenital heart disease Any heart condition or abnormality that a person was born with.
Congestive heart failure A common form of heart failure that results in a patient retaining excessive fluid, often leading to swelling of the legs and ankles and congestion in the lungs.
Coronary artery bypass A procedure used to reroute the blood supply around a blocked section of a coronary artery. Surgeons remove healthy blood vessels from another part of the body, such as a leg or the chest wall. Then they surgically attach the vessels to the diseased artery to let the blood flow around the blocked section.
Coronary artery disease A condition caused by thickening of the walls of the arteries that supply blood to the heart muscle. When these arteries become blocked, the heart is deprived of oxygen and can become damaged. Severe cases can result in heart attack.
Defibrillator A device that delivers pacing or an electric countershock to the heart when an abnormal rhythm is detected. A surgically implantable version is called a pacemaker.
Diastolic pressure The pressure of blood inside arteries when the heart is at rest. This is the bottom number in a blood pressure reading.
Dyspnea Difficult or labored breathing, often caused by heart conditions.
Edema An abnormal accumulation of fluid in body tissues.
Edema is common in the legs, ankles and lungs of people with heart failure.
Ejection fraction The amount of blood released during each contraction of the lower ventricle of the heart. It's usually expressed as a percentage: an ejection fraction of 60 percent means that 60 percent of the total amount of blood in the left ventricle is expelled with each heartbeat.
Electrocardiogram (EKG or ECG) A record of the electrical activity of the heart, allowing diagnosis of abnormal heart conditions.
Endocarditis Inflammation of the lining of the heart and the heart valves, usually due to bacterial infection.
Heart attack Sudden death of a portion of the heart muscle caused by a sudden decrease in blood supply to that area. Also known as myocardial infarction or MI.
Heart disease, ischemic The most prevalent form of heart disease, in which narrowed or blocked coronary arteries result in decreased blood supply.
Heart failure The inability of the heart to keep up with its workload. When someone has this condition, their heart can't pump enough blood to the lungs and the rest of the body. Heart failure is often a chronic condition that can be treated with medications, diet and other lifestyle changes, and in some cases, surgery.
Heart valve One of the four structures in the heart that control the flow of blood by opening and closing with each heartbeat. The valves permit the flow of blood in only one direction.
Hypertension The medical term for abnormally high blood pressure.
Hypotension Abnormally low blood pressure.
Left-ventricular assist device A mechanical pump used to aid the natural pumping action of the heart's left ventricle.
Left-ventricular heart failure Heart failure in which the left side of the heart must work harder to pump the same amount of blood. This type of heart failure usually causes breathing difficulties.
Myocardial infarction Sudden death of a portion of the heart muscle caused by a sudden decrease in blood supply to that area. See Heart Attack.
Myocarditis Inflammation of the heart muscle.
Potassium A mineral that, together with sodium and calcium, regulates the body's water balance, maintains normal heart rhythm, and is responsible for nerve impulse conduction and muscle contraction.
Pulmonary edema Fluid in lung tissues, often caused by congestive heart failure.
Right-ventricular heart failure Heart failure caused by damage to the right-side chambers of the heart, leading to decreased blood flow, and swelling in the hands, legs and abdomen.
Systolic pressure The pressure of blood inside arteries when the heart contracts. This is the top number in a blood pressure reading.
Vasoconstriction A narrowing of a blood vessel, causing decreased blood flow to a part of the body.
Vasodilator A medication that causes widening or relaxation of blood vessel walls. Examples include ACE inhibitors, angiotensin II receptor blockers, beta blockers, calcium channel blockers, natriuretic peptides and nitrates.
Ventricle One of the two lower chambers of the heart that receive blood from the atria (upper chambers). The right ventricle pumps blood to the lungs and the left ventricle pumps blood to the rest of the body.

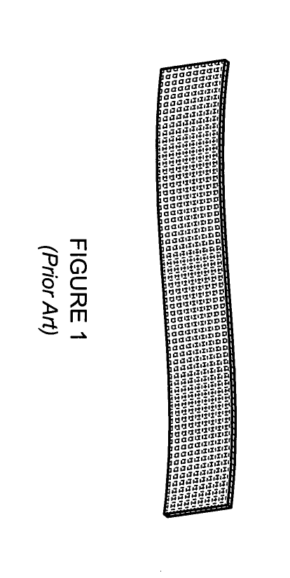

FIG. 1 illustrates an EAP segment according to the prior art. The EAP can be easily deformed upon the application of low voltage (approximately 1-3.5 V). Deflection varies linearly at low applied voltages (<1 V) with nonlinear behavior observed at higher voltages. At the linear range the EAP deforms at a rate of about 20-35 degrees/volt. The magnitude of deflection of the EAP strip (measured in degrees of deflection) is similar in both directions (upon reversing the polarity of the electrical field). This suggests that the EAP surfaces have similar conductivity and that the EAP composition is reasonably uniform. However, the EAP strip can at times deflect significantly more in one direction and the change in deflection variation with voltage is non-linear. In the above cases resistance measurements can be used to verify if the less conductive side of the EAP is contact with the negative electrode which would result in the observed reduction in bending. Such a behavior is believed to be due to either loss of positive counter ions in the matrix (due to repeated soaking of the EAP in water) or imperfections in the EAP conductive surface. For the specific EAP tested in this illustration the change in deflection with applied voltage was greater above about 2.5 V. In other words, at higher voltages the EAP the applied voltage causes a greater deflection per volt than at low voltages.

The EAP performs well when immersed in water. The deflection is somewhat less than in air given the additional work that the EAP strip has to perform in order to displace water as it deforms. The deflection of the EAP in water is more consistent and the electromechanical response does not change significantly over 20-30 minutes. In contrast, the performance of the EAP in air declines over time, requiring re-wetting of the EAP after a period of about 3-5 minutes.

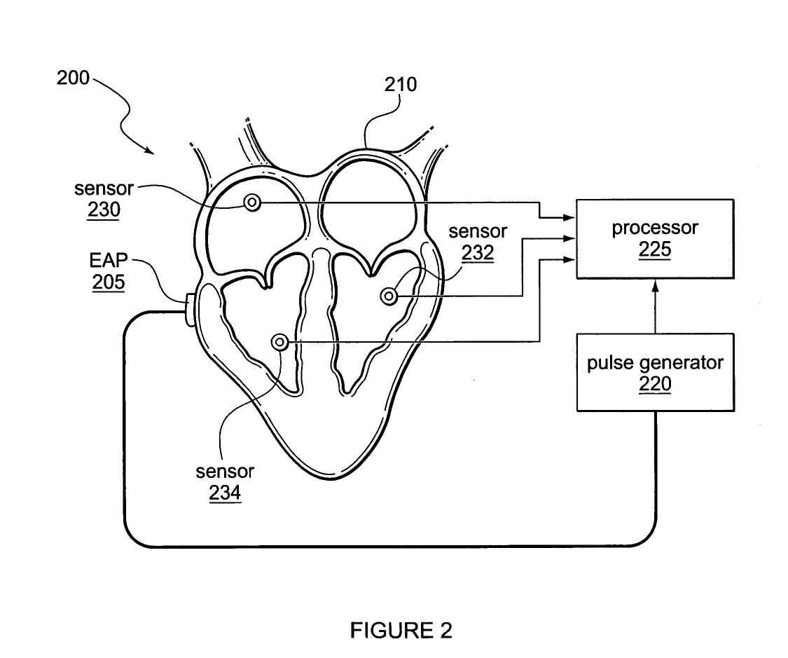

FIG. 2 illustrates a cardiac contractile augmentation device comprising an EAP segment that is applied to a selected area of the heart according to an embodiment of the present invention.

According to an embodiment of the present invention, a cardiac contractile assist device (CCAD) 200 comprising an EAP segment 205 is configured as a “patch” that provides reinforcement of the heart wall at a localized area of the heart 210, such as a cardiac aneurysm or at an area of the myocardium which has been damaged due to myocardial infarction, while providing contractile augmentation of the local area to which it is applied. The size of the patch is selected to cover an area of the epicardial surface of the heart 200 in need of reinforcement without completely surrounding the circumference of the heart. The EAP segment 205 is connected to pulse generator 220. Pulse generator 220 is connected to, and responsive to, processor 225. According to an embodiment of the present invention, processor 225 comprises instructions that cause pulse generator 220 to apply pulses to the EAP segment 205 according at a predetermined interval.

Optionally, processor 225 receives data from sensors 230, 232, and 234 that is indicative of the status of the heart. By way of illustration and not as a limitation, sensors are used to provide a processor data indicative of the state of the heart and the need for contractile augmentation. Sensors may be attached to the heart 210 or may be integrated into a CCAD. Sensors may be used to detect the activation of the heart 210 or to detect a measure of heart activity. By way of illustration and not as a limitation, sensing may be directed to metabolic demand, the QT interval of the ECG cycle, the right ventricular systolic pressure blood oxygen saturation, the respiration rate partial pressure of carbon dioxide in the blood, blood temperature and pre-ejection period.

A CCAD patch can be prepared from the biomedical materials described above. In a preferred embodiment, the patch is an open mesh material.

A CCAD patch can be applied to the epicardial surface of the heart 210 over or under the parietal pericardium. A patch is typically applied to the epicardial surface by suturing around the periphery of the patch. The peripheral edge of the patch can include a thickened “ring” or other reinforcement to enhance the strength of the patch at the point of suture attachment to the epicardium. Generally, a patch is applied to the epicardium through a thoracotomy or other incision providing sufficient exposure of the heart.

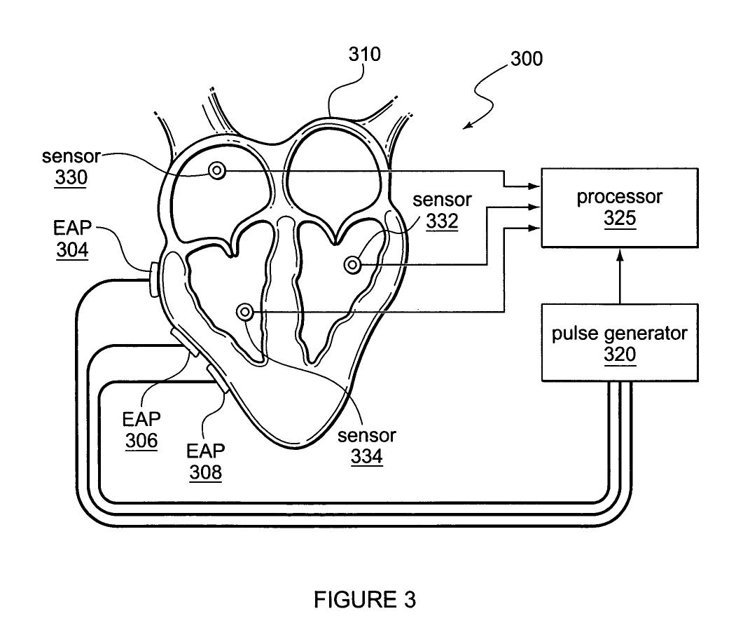

FIG. 3 illustrates a cardiac contractile augmentation device comprising a plurality of EAP segments that is applied to a selected area of the heart according to an embodiment of the present invention.

Referring to FIG. 3, a cardiac contractile assist device (CCAD) 300 comprising a plurality of EAP segments 304, 306 and 308 is configured as a “patch” that provides reinforcement of the heart wall of the heart 310 at a localized area, such as a cardiac aneurysm or at an area of the myocardium which has been damaged due to myocardial infarction, while providing contractile augmentation of the local area to which it is applied. The size of the patch is selected to cover an area of the epicardial surface of the heart 310 in need of reinforcement without completely surrounding the circumference of the heart 310. The EAP segments 304, 306 and 308 are connected to pulse generator 320. Pulse generator 320 is connected to, and responsive to, processor 325. According to an embodiment of the present invention, processor 325 comprises instructions that cause pulse generator 320 to apply pulses to the EAP segments 304, 306 and 308 according at a predetermined interval.

According to an embodiment of the present invention, pulse generator 320 comprises a sufficient number of independent outputs to control EAP segments 304, 306 and 308 independently.

Optionally, processor 325 receives data from sensors 330, 332, and 334 that is indicative of the status of the heart 310. By way of illustration and not as a limitation, sensors 330, 332, and 334 provide a processor data indicative of the state of the heart 310 and the need for contractile augmentation. Sensors 330,332, and 334 may be attached to the heart or may be integrated into CCAD 330. Sensors 330, 332, and 334 may be used to detect the activation of the heart 310 or to detect a measure of heart activity. By way of illustration and not as a limitation, sensing may be directed to metabolic demand, the QT interval of the ECG cycle, the right ventricular systolic pressure blood oxygen saturation, the respiration rate partial pressure of carbon dioxide in the blood, blood temperature and pre-ejection period.

According to an embodiment of the present invention, each segment of the plurality of EAP segments 304, 306 and 308 is independently energized. Pulse generator 320 under control of a processor 325 is connected to the individual segments 304, 306 and 308. The processor 325 causes the pulse generator 320 to sequentially pulse the individual segments in the direction of a normal contraction of the cardiac tissue. In this way, the CCAP 300 provides contractile augmentation to a chamber of the heart in a pattern that is equivalent to a concentric contraction of normal heart muscle.

A CCAD patch can be prepared from the biomedical materials described above. In a preferred embodiment, the patch is an open mesh material.

A CCAD patch can be applied to the epicardial surface of the heart over or under the parietal pericardium. A patch is typically applied to the epicardial surface by suturing around the periphery of the patch. The peripheral edge of the patch can include a thickened “ring” or other reinforcement to enhance the strength of the patch at the point of suture attachment to the epicardium. Generally, a patch is applied to the epicardium through a thoracotomy or other incision providing sufficient exposure of the heart 310.

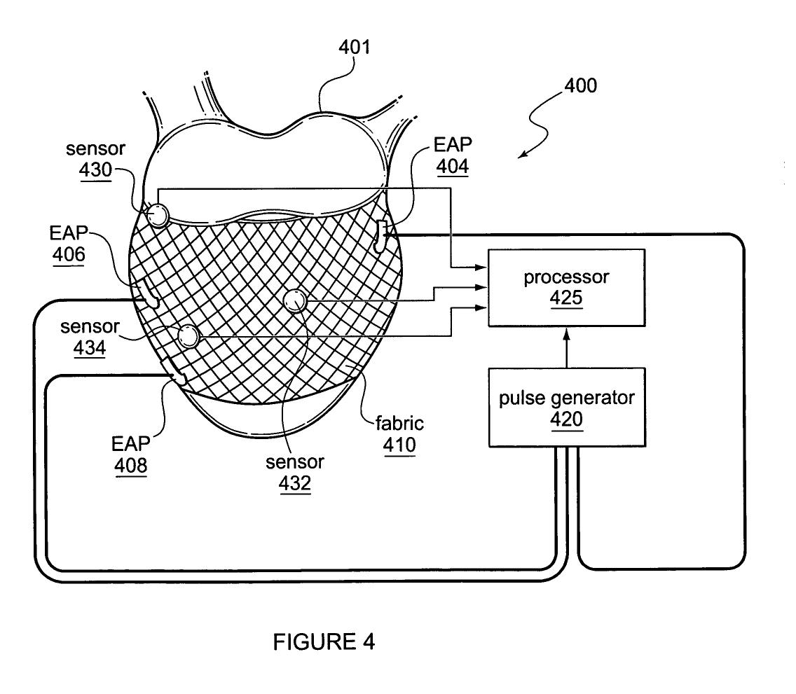

FIG. 4 illustrates a cardiac contractile augmentation device configured as a cardiac wrap and comprising at least one EAP segment according to an embodiment of the present invention.

Referring to FIG. 4, a cardiac contractile augmentation device (CCAD) 400 comprises EAP segments 404, 406 and 408 integrated with a reinforcing fabric 410, a pulse generator 420, a processor 425, sensors 430, 432 and 434. The CCAD in this embodiment (referred to herein as a “cardiac wrap”) circumscribes the heart 401. This embodiment of the present invention is particularly suited for use in cardiomyopathies where abnormal dilation of one or more chambers of the heart 401 is a component of the disease.

As used herein, “cardiac chamber” refers to the left or right atrium or the left or right ventricle. The term “myocardium” refers to the cardiac muscle comprising the contractile walls of the heart 401. The term “endocardial surface” refers to the inner walls of the heart 401. The term “epicardial surface” refers to the outer walls of the heart 401.

The heart 401 is enclosed within a double walled sac known as the pericardium. The inner layer of the pericardial sac is the visceral pericardium or epicardium. The outer layer of the pericardial sac is the parietal pericardium.

According to an embodiment of the present invention, the CCAD 400 limits the outward expansion of the heart wall during diastolic chamber filling beyond a desirable size. The expansion constraint applied to the heart 401 by a CCAD 400 is determined by processor 425 that applies an algorithm to data from sensors 430, 432, and 434 that acquire metrics indicative of the state of the heart and the need for contractile augmentation. Sensors may be attached to the heart or may be integrated into a CCAD. Sensors may be used to detect the activation of the heart 401 or to detect a measure of heart activity. By way of illustration and not as a limitation, sensing may be directed to metabolic demand, the QT interval of the ECG cycle, the right ventricular systolic pressure blood oxygen saturation, the respiration rate partial pressure of carbon dioxide in the blood, blood temperature and pre-ejection period.

Sensor data are used by processor 425 to activate EAG segments 404, 406 and 408 to control the tension applied to the heart 401 during diastole by CCAD 400. Separately or simultaneously, EAG segments 404, 406 and 408 may be used to provide contractile augmentation to a portion of the heart 401 in contact with CCAD 400. In this embodiment of the present invention, processor 425 applies algorithms to determine which EAG segments 404, 406 and 408 are to be energized by pulse generator 420 and the timing of the pulses applied to those segments. According to an embodiment of the present invention, pulse generator 420 comprises a sufficient number of independent outputs to control EAP segments 404, 406 and 408 independently.

The reinforcing fabric 410 of CCAD 400 is made from a biomedical material which can be applied to the epicardial surface of the heart 401. As used herein, a “biomedical material” is a material which is physiologically inert to avoid rejection or other negative inflammatory response. A CCAD 400 can be prepared from an elastic or substantially non-elastic biomedical material. The biomedical material can be inflexible, but is preferably sufficiently flexible to move with the expansion and contraction of the heart 401 without impairing systolic function. The biomedical material should, however, constrain cardiac expansion, during diastolic filling of the heart 401, to a predetermined size. Examples of suitable biomedical materials include perforate and non-perforate materials. Perforate materials include, for example, a mesh such as a polypropylene or polyester mesh. Non-perforate materials include, for example, silicone rubber.

A biomedical material suitable for a device of the invention generally has a lower compliance than the heart wall. Even though the biomedical material is less compliant than the heart wall, some limited expansion of an elastic biomedical material can occur during cardiac filling.

In an alternative embodiment, the biomedical material can be substantially non-elastic. According to this embodiment, the term “substantially non-elastic” refers to a material which constrains cardiac expansion during diastole at a predetermined size, but which has substantially no elastic properties.

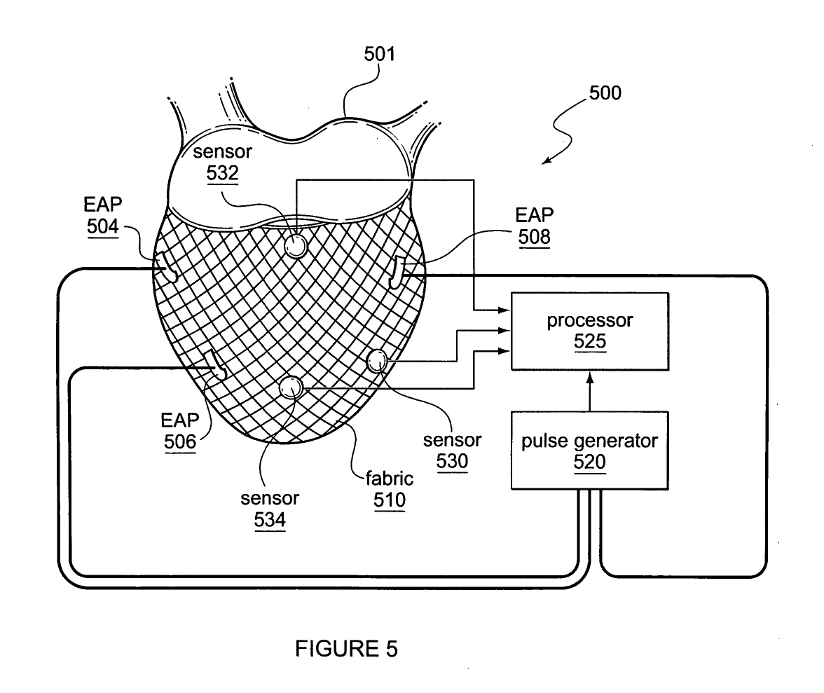

FIG. 5 a cardiac contractile augmentation device configured as a cardiac envelope and comprising at least one EAP segment according to an embodiment of the present invention.

Referring to FIG. 5, a CCAD 500 is configured as a cardiac envelope that substantially contains the epicardial surface of the heart. When applied to the heart 501, a CCAD envelope can be placed over or under the parietal pericardium. Referring to FIG. 5, a cardiac contractile augmentation device (CCAD) 500 comprises EAP segments 504, 506 and 508 integrated with a reinforcing fabric 510, a pulse generator 520, a processor 525, sensors 530, 532 and 534. While FIG. 5 illustrates three EAP segments and three sensors, the present invention is not so limited. The number of EAP segments and sensors maybe adjusted upwards or downwards to suit the requirements of a particular patient. The behavior of the cardiac envelope is similar to that described above in reference to the cardiac wrap. However, the cardiac envelope constrains the heart 501 in all directions. Additionally, the cardiac envelope permits that application of contractile augmentation to the chambers of the heart 501 so in concert with the normal the rhythmic beating of the heart 501.

A CCAD 500 envelope applied to the epicardium is fitted to an initial size to limit cardiac expansion. According to an embodiment of the present invention, the initial size of the cardiac envelope may be established by passively by the elasticity of the envelope material or by applying a bias voltage to one or more of the EAP segments 504, 506 and 508. In either of these embodiments, the initial size of the cardiac envelope circumferentially constrains cardiac expansion during diastolic filling of the heart. In practice, for example, a physician could measure cardiac output and adjust the envelope size to an optimal size for a desired effect.

As the need for restraint of the heart changes, the heart envelope may be adjusted electrically to suit the requirements of the patient. For example, the initial size of the cardiac envelope may be established to apply greater restraint to the heart at the beginning of therapy with the intention of applying less restraint as the therapy progresses. If the EAP segments 504, 506 and 508 of the cardiac envelope are biased in a contracted state, the bias voltage may be reduced over time to allow the envelope size to increase. Alternatively, the initial size of the cardiac envelope may be established to apply less restraint to the heart at the beginning of therapy with the intention of applying greater restraint as the therapy progresses. In this case, the EAPs segments 504, 506 and 508 may be biased so as to as maximize the size of the cardiac envelope at the initially stages of therapy.

As will be appreciated by those skilled in the art, the EAP segments 504, 506 and 508 may be configured within the cardiac envelope of CCAD 500 to perform different tasks. For example, certain EAP segments may be used to establish an initial size of the cardiac envelope. When the range of adjustment of these EAP segments has been met, other EAP segments may then be energized to permit further adjustment of the cardiac envelope. Still other EAP segments may be assigned the task of providing contractile augmentation. Control of the various EAP segments is established by instructions executed by the processor and carried out by the pulse generator. According to an embodiment of the present invention, pulse generator 520 comprises a sufficient number of independent outputs to control each EAP segment independently.

In one-embodiment, the CCAD envelope is a cone-shaped tube, having a base broader than the apex, which generally conforms to the external geometry of the heart. When applied to the epicardial surface of the heart, the base of the envelope is oriented towards the base of the heart, and the apex of the envelope is oriented towards the apex of the heart. Typically, the base of the envelope includes an opening for applying the envelope by passing the envelope over the epicardial surface of the heart. The apical end of the envelope can be a continuous surface which covers the apex of the heart. Alternatively, the apex of the envelope can have an opening through which the apex of the heart protrudes.

In one embodiment, the CCAD envelope can be secured to the epicardium by a securing arrangement mounted at the base of the envelope. A suitable securing arrangement includes, or example, a circumferential attachment device, such as a cord, suture, band, adhesive or shape memory element which passes around the circumference of the base of the envelope. The ends of the attachment device can be fastened together to secure the envelope in place. Alternatively, the base of the envelope can be reinforced for suturing the base of the envelope to the epicardium.

The biomedical material of the invention can be radioluscent or radiopaque. In one embodiment, the material of the envelope can be made radiopaque by inclusion of radiopaque markers for identification of the outside surface of the heart. As used herein, radiopaque means causing the CCAD to be visible on x-ray or fluoroscopic viewing. Suitable radiopaque markers include, for example, platinum wires, titanium wires and stainless steel wires.

A CCAD according to the present disclosure provides a new method for the treatment of cardiac disease. As used herein, cardiac disease includes diseases in which dilation of one of the chambers of the heart is a component of the disease. Examples include heart failure or cardiomyopathy. Heart failure can occur as a result of cardiac dilation due to ventricular hypertrophy or secondary to, for example, valvular incompetency, valvular insufficiency or valvular stenosis. Cardiomyopathy, according to the invention, can be primary or secondary to infection, ischemia, metabolic disease, genetic disorders, etc.

It is foreseen that constraint of cardiac expansion by a device of the invention can provide reduced cardiac dilation. Reduced cardiac dilation can cause reduction in the problems associated with cardiac dilation such as arrhythmias and valvular leakage. As reduction of cardiac dilation occurs, selective reduction of the predetermined size of the envelope also provides continued reinforcement for the size reduced heart.

A CCAD envelope can also be used to measure cardiac performance. According to this embodiment, the CCAD envelope comprises strain measurement devices that can be used to gather data indicative of cardiac performance. These data may be reported to the processor and retained in memory. A communications channel from the processor-memory components to an external monitoring device allows evaluation of cardiac performance and may assist in adjusting the algorithms used to control the CCAD envelope.

A CCAD as described herein can be applied to the epicardium of a heart through a thoracotomy or through a minimally invasive procedure. For a minimally invasive procedure a CCAD placement tool can be used to apply the CCAD over the epicardium of the heart through a thorascopic incision. According to this embodiment, a CCAD placement tool includes a cannula, a stiff rod or wire and a guide tube. For placement of a CCAD, the wire is threaded through the guide tube which is passed around the circumference of the base of the envelope. The CCAD with wire and guide tube passed through the base opening are then passed into the cannula. The cannula is of sufficient length and diameter to enclose the CCAD, wire and guide tube during passage of the placement tool through a thorascopic incision. The placement tool is passed into the thoracic cavity and positioned at a point near the apex of the heart. When in position, the wire and guide tube are pushed out of the cannula away from the operator. Once outside the cannula, the wire and guide tube sufficiently expand the opening of the base of the CCAD envelope to pass over the epicardial surface of the heart. When the CCAD envelope is in position over the epicardial surface, the wire, guide tube and cannula can be removed. A second incision can then be made to provide access for suitable surgical instruments to secure or adjust the size of the CCAD.

As previously noted, a CCAD may be combined with pacing to enhance the contraction of the heart and to aid in controlling and reversing the affects of adverse remodeling. In an embodiment of the present application, CCAD is used in conjunction with biphasic. In cardiac muscle, the muscle fibers are interconnected in branching networks that spread in all directions through the heart. When any portion of this net is stimulated, a depolarization wave passes to all of its parts and the entire structure contracts as a unit. Before a muscle fiber can be stimulated to contract, its membrane must be polarized. A muscle fiber generally remains polarized until it is stimulated by some change in its environment. A membrane can be stimulated electrically, chemically, mechanically or by temperature change. The minimal stimulation strength needed to elicit a contraction is known as the threshold stimulus. The maximum stimulation amplitude that may be administered without eliciting a contraction is the maximum subthreshold amplitude.

Where the membrane is stimulated electrically, the impulse amplitude required to elicit a response is dependent upon a number of factors. First, is the duration of current flow. Since the total charge transferred is equal to the current amplitude times the pulse duration, increased stimulus duration is associated with a decrease in threshold current amplitude. Second, the percentage of applied current that actually traverses the membrane varies inversely with electrode size. Third, the percentage of applied current that actually traverses the membrane varies directly with the proximity of the electrode to the tissue. Fourth, the impulse amplitude required to elicit a response is dependent upon the timing of stimulation within the excitability cycle.

In an embodiment of the present invention, biphasic stimulation is applied to the heart, wherein both cathodal and anodal pulses are administered. According to one aspect of this embodiment, this stimulation is administered to the myocardium in order to enhance myocardial function. According to a further aspect of this invention, this stimulation is administered directly to the cardiac tissue or to the cardiac blood pool. This enables cardiac stimulation without the necessity of placing electrical leads in intimate contact with cardiac tissue.

The biphasic stimulation comprises a first and second stimulation phase, with each stimulation phase having a polarity, amplitude, shape and duration. In one embodiment of the present invention, the first and second phases have differing polarities. In another embodiment of the present invention, the two phases are of differing amplitude. In yet another embodiment of the present invention, the two phases are of differing duration. In still another embodiment of the present invention, the first phase is in a chopped wave form. In a further embodiment of the present invention, the amplitude of the first phase is ramped. In yet another embodiment of the present invention the first phase is administered over 200 milliseconds post heart beat; i.e., greater than 200 milliseconds after completion of a cardia beating/pumping cycle. In still another embodiment of the present invention, the first phase of stimulation is an anodal pulse at maximum subthreshold amplitude for a long duration, and the second phase of stimulation is a cathodal pulse of short duration and high amplitude. It is noted that the aforementioned embodiments can be combined in differing fashions. It is also noted that these embodiments are intended to be presented by way of example only, and are not limiting.

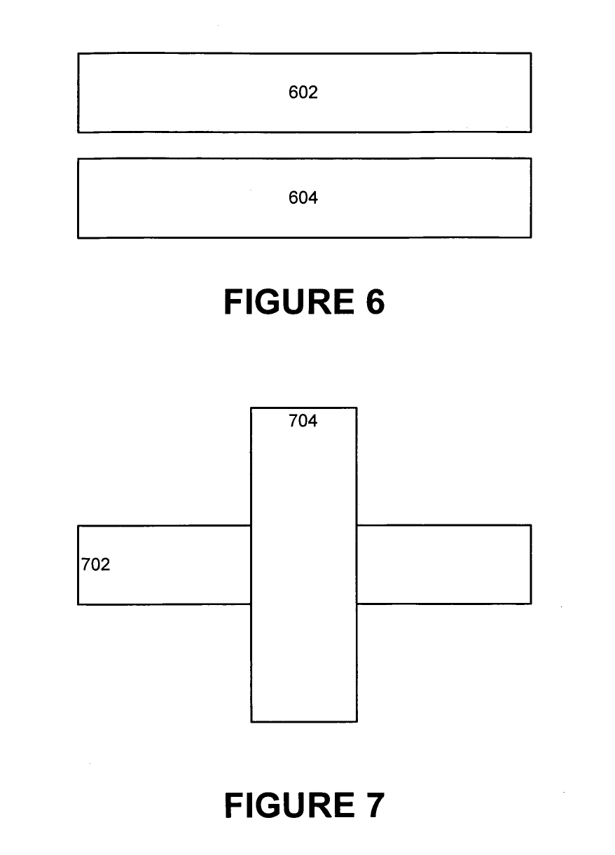

The CCADs described in the context of FIGS. 2, 3, 4 and 5 are illustrated as using EAP segments that are of single-layer constructions. However, this description is not meant as a limitation. EAP segments may be arranged in various configurations without departing from the scope of the present invention. By way of illustration and not as a limitation, FIG. 6 illustrates two EAP segments 602 and 604 arranged back-to-back according to an embodiment of the present invention. The EAP segments are then energized with different polarities such that one relaxes the other contracts along the same axis. Again, by way of illustration and not as a limitation, FIG. 7 illustrates two EAP segments 702 and 704 that are overlapped according to an embodiment of the present invention. In this embodiment, the two EAP segments are energized so as to provide a global contraction pattern.

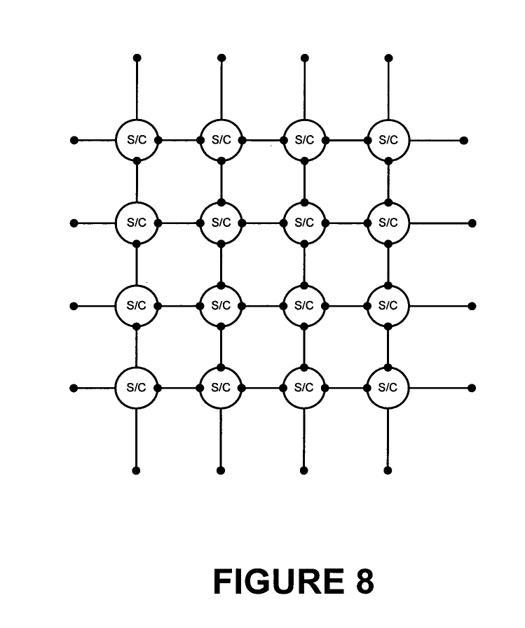

Referring now to FIG. 8 a network of dual use (sensing and contraction) EAPs is illustrated according to an embodiment of the present invention. In this embodiment, a portion of the fabric of the CCAD is illustrated. Dual use EAPs such as those disclosed in U.S. Pat. No. 6,689,288 to St. Clair et al. (which is incorporated herein by reference for all purposes) discloses a Polymeric blend which achieves dual function sensing and actuation. Using EAPs of this polymeric type, a CCAD fabric is constructed that allows sensing of electrical activity of the heart to occur over the entire heart muscle. Sensing signals are sent to a processor that notes the areas in need of assistance and targeted contractions are then delivered. In this figure, while circular cross points are noted, in practice these intersections are merely the intersection of crossing EAP fibers or strips. However, this structure gives better locational awareness of areas of the heart that may need assistance.

Once a trouble spot (area) is sensed, current can be applied to that section of the CCAD to give targeted contraction where needed. In this way, specific areas of heart muscle, that might require assistance, can be isolated.

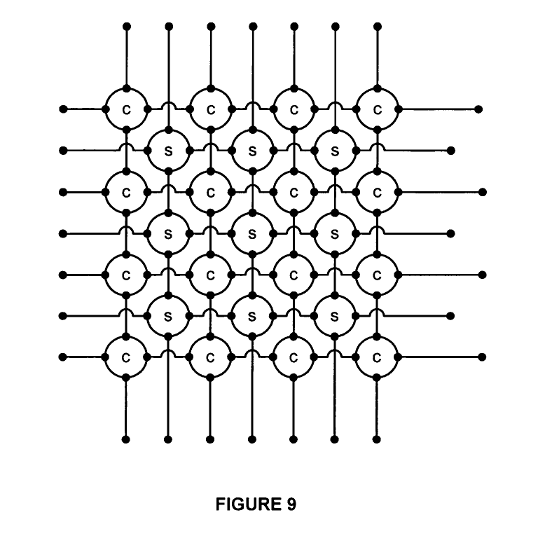

Referring now to FIG. 9, two separate networks of sensing and contracting EAPS is illustrated. In this instance, the sensing network (indicated by the “S” at the cross points) identified trouble areas of the heart muscle. Signals are then sent to a processor for subsequent deliver of current to the contraction network (indicated by “C” at the cross points) so that targeted contractions can be delivered to specific areas in need of assistance.

In the various instances noted above, it is a further embodiment of the present invention to deliver biphasic stimulation to the heart muscle as well, to the extent necessary, to aid in the physical contraction needed. Thus, electrical stimulation may potentially be decreased when used together with physical contraction thus minimizing any detrimental impact on the heart muscle.

While embodiments of the present invention have been describe in relationship to the heart, the present invention is not so limited. Contractile augmentation and stimulation as provided by the present invention may be desirable to stimulate striated muscle tissue and smooth muscle. By way of illustration and not as a limitation, embodiments of the present invention may be applied to those muscles responsible for the movements that force food through the digestive tube, constrict blood vessels and empty the urinary bladder.

A cardiac contractile augmentation device and method therefor have been described. It will be understood by those skilled in the art that the present invention may be embodied in other specific forms without departing from the scope of the invention disclosed and that the examples and embodiments described herein are in all respects illustrative and not restrictive. Those skilled in the art of the present invention will recognize that other embodiments using the concepts described herein are also possible. Further, any reference to claim elements in the singular, for example, using the articles “a,” “an,” or “the” is not to be construed as limiting the element to the singular. Moreover, a reference to a specific time, time interval, and instantiation of scripts or code segments is in all respects illustrative and not limiting.

What is claimed is:

1. A cardiac contractile augmentation device (CCAD) comprising: an electroactive polymer (EAP) linear segment attached along its length to a selected area of a heart, wherein the EAP segment may be energized during systole to augment the contractile strength of the heart.

2. The CCAD of claim 1, wherein the selected area of the heart is a ventricle.

3. The CCAD of claim 1, wherein the selected area of the heart is an atrium.

4. The CCAD of claim 1 further comprising:
a pulse generator adapted to:
generate a contraction electrical signal that coincides with an excitatory pulse directed to the heart at systole; and
generate a relaxation signal that coincides with a refractory period of the heart at diastole; and wherein
the EAP segment is adapted to:
receive the contraction signal from the pulse generator;
in response to the contraction signal from the pulse generator contract along its length thereby augmenting a contraction of the selected area of the heart to which the EAP segment is attached; and
in response to the relaxation signal from the pulse generator, return to a relaxed state.

5. The CCAD of claim 4 further comprising a sensor and a processor and wherein:
the sensor is adapted to acquire sensor data indicative of the state of the heart;
the processor is adapted to determine from the sensor data the need for contractile augmentation of the selected area; and
the pulse generator is responsive to commands from the processor to provide contraction and relaxation electrical signals.

6. The CCAD of claim 5, wherein the sensor data comprises at least one measure of the state of the heart selected from the group consisting of a measure of metabolic demand, an occurrence of the QT interval of the ECG cycle, a measure of a right ventricular systolic pressure blood oxygen saturation, a measure of a respiration rate partial pressure of carbon dioxide in the blood, a measure of blood temperature, and an occurrence of a pre-ejection period.

7. The CCAD of claim 4, wherein the contraction electrical signal comprises a chopped waveform with an asymmetric duty cycle.

8. The CCAD of claim 4, wherein the excitatory pulse is generated by the heart.

9. The CCAD of claim 5, wherein the excitatory pulse is generated by a heart stimulation device.

10. The CCAD of claim 9, wherein the heart stimulation devices comprises:
a left ventricular electrode group, wherein the left ventricular electrode groups comprise LV electrodes attached to the left ventricle at increasing distances from the AV node; and
a right ventricular electrode group, wherein the right ventricular electrode group comprises RV electrodes attached to the left ventricle at increasing distances from the AV node, and
wherein, the heart stimulation device is further adapted to:
attach to the LV and RV electrodes;
generate a timing signal coincident with a refractory period of the heart; and
in response to the timing signal, send pulses to the LV and RV electrodes sequenced such that an initial pulse arrives at an LV electrode and at an RV electrode nearest the AV junction and subsequent pulses arrive at an LR and at an RV electrode progressively further from the AV junction.

11. A cardiac contractile augmentation device (CCAD) comprising:
two or more electroactive polymer (EAP) linear segments each attached along its length to a selected area of a heart, wherein selected EAP segments may be energized during systole to augment the contractile strength of the heart.

12. The CCAD of claim 11 wherein the selected area of the heart is a ventricle.

13. The CCAD of claim 11 wherein the selected area of the heart is an atrium.

14. The CCAD of claim 11 further comprising:
a pulse generator adapted to:
generate a contraction electrical signal that coincides with an excitatory pulse directed to the heart at systole; and
generate a relaxation signal that coincides with a refractory period of the heart at diastole; and wherein
an EAP segment is adapted to:
receive the contraction signal from the pulse generator;
in response to the contraction signal from the pulse generator contract along its length thereby augmenting a contraction of a selected area of the heart to which the EAP segment is attached; and
in response to the relaxation signal from the pulse generator, to return a relaxed state.

15. The CCAD of claim 14 further comprising a sensor and a processor and wherein:
the sensor is adapted to acquire data indicative of the state of the heart;
the processor is adapted to determine from the sensor data the need for contractile augmentation of the selected area; and
the pulse generator is responsive to commands from the processor to provide contraction and relaxation electrical signals to the selected EAD segments.

16. The CCAD of claim 15, wherein the sensor data comprises at least one measure of the state of the heart selected from the group consisting of a measure of metabolic demand, an occurrence of the QT interval of the ECG cycle, a measure of a right ventricular systolic pressure blood oxygen saturation, a measure of a respiration rate partial pressure of carbon dioxide in the blood, a measure of blood temperature, and an occurrence of a pre-ejection period.

17. The CCAD of claim 15 wherein:
the processor is further adapted to generate a sequence of commands timed to coincide with a normal contraction of the selected area of the heart;
the pulse generator is adapted send a contraction electrical signal sequentially to selected EAD segments in response to the sequence of commands from the processor.

18. The CCAD of claim 17 wherein the contraction electrical signal comprises a chopped waveform with an asymmetric duty cycle.

19. The CCAD of claim 14, wherein the excitatory pulse is generated by the heart.

20. The CCAD of claim 14, wherein the excitatory pulse is generated by a heart stimulation device.

21. The CCAD of claim 20, wherein the heart stimulation device comprises:
a left ventricular electrode group, wherein the left ventricular electrode groups comprise LV electrodes attached to the left ventricle at increasing distances from the AV node; and
a right ventricular electrode group, wherein the right ventricular electrode group comprises RV electrodes attached to the left ventricle at increasing distances from the AV node, and
wherein, the heart stimulation device is further adapted to:
attach to the LV and RV electrodes;
generate a timing signal coincident with a refractory period of the heart; and
in response to the timing signal, send pulses to the LV and RV electrodes sequenced such that an initial pulse arrives at an LV electrode and at an RV electrode nearest the AV junction and subsequent pulses arrive at an LR and at an RV electrode progressively further from the AV junction.

22. An active cardiac envelope (ACE) comprising:
a biomedical material that can be applied to the epicardial surface of the heart that expands to a predetermined size, the predetermined size selected to constrain cardiac expansion beyond a predetermined limit; and
an augmentation EAP segment integrated with the biomedical material, wherein the augmentation EAP segment may be energized during systole to augment the contractile strength of the heart.

23. The ACE of claim 22 wherein the ACE surrounds the epicardial surface of the heart and circumferentially constrains cardiac expansion.

24. The ACE of claim 22 wherein the envelope has a base end, the base end having an opening for applying the envelope to the epicardial surface of the heart by passing the envelope over the epicardial surface of the heart such that when applied to the epicardial surface, the base end of the envelope is oriented toward the base of the heart.

25. The ACE of claim 22 wherein the envelope has an apex end such that when the envelope is applied to the epicardial surface, the apex end is oriented towards the apex of the heart.

26. The ACE of claim 24 wherein the base end of the envelope further comprises a securing arrangement for securing the envelope to the epicardial surface of the heart.

27. The ACE of claim 22 further comprising a sensor, a processor, and a pulse generator and wherein:
the sensor is adapted to acquire sensor data indicative of the state of the heart;
the processor is adapted to determine from the sensor data the need for contractile augmentation of the selected area; and
the pulse generator is adapted to respond to a command from the processor to provide a contraction signal to the augmentation EAP segment.

28. A cardiac contractile augmentation device (CCAD) comprising:
an electroactive polymer (EAP) linear segment attached along its length to a selected area of a heart, wherein the EAP segment may be energized during systole to augment the contractile strength of the heart;
a EAP pulse generator adapted to:
generate a contraction electrical signal that coincides with the application of a biphasic pacing pulse from a heart stimulation device connected to the heart; and
generate a relaxation signal that coincides with a refractory period of the heart at diastole; and wherein
the EAP linear segment is adapted to:
receive the contraction signal from the pulse generator;
in response to the contraction signal from the pulse generator contract along its length thereby augmenting a contraction of the selected area of the heart to which the EAP segment is attached; and
in response to the relaxation signal from the EAP pulse generator, return to a relaxed state.

29. The CCAD of claim 28 further comprising a sensor and a processor and wherein:
the sensor is adapted to acquire sensor data indicative of the state of the heart;
the processor is adapted to determine from the sensor data the need for contractile augmentation of the selected area; and
the EAP pulse generator is responsive to commands from the processor to provide contraction and relaxation electrical signals.

30. The CCAD of claim 29, wherein the sensor data comprises at least one measure of the state of the heart selected from the group consisting of a measure of metabolic demand, an occurrence of the QT interval of the ECG cycle, a measure of a right ventricular systolic pressure blood oxygen saturation, a measure of a respiration rate partial pressure of carbon dioxide in the blood, a measure of blood temperature, and an occurrence of a pre-ejection period.

31. The CCAD of claim 28, wherein the contraction electrical signal comprises a chopped waveform with an asymmetric duty cycle.

32. The CCAD of claim 28, wherein the biphasic pacing pulse comprises:
a first stimulation phase with a first phase polarity, a first phase amplitude, a first phase shape and a first phase duration for preconditioning the myocardium to accept subsequent stimulation;
a second stimulation phase with a polarity opposite to the first phase polarity, a second phase amplitude that is larger in absolute value than the first phase amplitude, a second phase shape and a second phase duration; and
wherein, the first stimulation phase and the second stimulation phase are applied in sequence to cardiac tissue.

33. The CCAD of claim 32, wherein the first phase polarity is positive.

34. The CCAD of claim 32, wherein the first phase amplitude is ramped from a baseline value to a second value.

35. The CCAD of claim 34, wherein the second value is at a maximum subthreshold amplitude.

36. The CCAD of claim 32, wherein the first phase duration is at least as long as the second phase duration.

37. An active cardiac fabric (ACF) system comprising:
a pulse generator, wherein the pulse generator is responsive to a processor;
a active cardiac fabric comprising a network of EAP sensors and a network of EAP contractile devices that can be applied to a selected segment of the heart;
wherein an EAP sensor is adapted for:
sensing electrical activity of the heart; and
generating an electrical activity signal in response to the sensed activity; and
wherein an EAP contractile device is adapted for contracting in response to a contraction signal sent by the pulse generator; and
wherein the processor is adapted for:
receiving the electrical activity signal from the EAP sensor;
determining whether the heart requires contractile augmentation at location proximate to the EAP contractile device; and
sending a contraction instruction to the pulse generator to generate a contraction signal if the heart requires contractile augmentation at the location proximate to the EAP contractile device, wherein the pulse generator sends the contraction signal to the EAP contractile device.

38. The ACF system of claim 37, wherein the selected area of the heart is a ventricle.

39. The ACF system of claim 37, wherein the selected area of the heart is an atrium.

40. The ACF system of claim 37, wherein the contraction signal is sent during systole.

41. An active cardiac fabric (ACF) system comprising:
a pulse generator, wherein the pulse generator is responsive to a processor;
an active cardiac fabric comprising a network of dual function EAP device that can be applied to a segment of the heart;
wherein a dual function EAP device is adapted for:
sensing electrical activity of the heart;
generating an electrical activity signal in response to the sensed activity; and
contracting in response to a contraction signal sent by the pulse generator; and
wherein the processor is adapted for:
receiving the electrical activity signal;
determining whether the heart requires contractile augmentation at location proximate to the dual function EAP; and
sending a contraction instruction to the pulse generator to generate a contraction signal if the heart requires contractile augmentation at the location proximate to the dual function EAP, wherein the pulse generator sends the contraction signal to the dual function EAP.

42. The ACF system of claim 41, wherein the selected area of the heart is a ventricle.

43. The ACF system of claim 41, wherein the selected area of the heart is an atrium.

44. The ACF system of claim 41 wherein the contraction signal is sent during systole.

45. The ACF system of claim 41, wherein the dual function EAP comprises a blended polymer.
